(19) United States
(12) Patent Application Publication
(10) Pub. No.: US 2007/0149950 A1
(43) Pub. Date: Jun. 28, 2007

(54) Thin wall surgical irrigation tubing with longitudinal reinforcements

(75) Inventors: James T. Perkins, St. Charles, MO (US); Michael Versemann, St. Charles, MO (US); Jeffery A. Knight, Maryland Heights, MO (US)

Correspondence Address: Michael L. Smith;Bausch & Lomb Incorporated, One Bausch & Lomb Place, Rochester, NY, 14604, US

(73) Assignee: Bausch & Lomb Incorporated

(21) Appl. No.: 11/708,256

(22) Filed: Feb. 20, 2007

Related U.S. Application Data
(63) Continuation-in-part of application No. 11/186,734, filed on Jul. 21, 2005.

(51) Int. Cl. A61M 25/00

(52) U.S. Cl. 604/524; 604/264

(57) ABSTRACT

A length of ophthalmic surgical irrigation tubing 14 includes an inner lumen 18 for transporting fluid from a source 24 to a surgical handpiece 26. The tubing 14 has an outer surface 20 including reinforcement structure 22 formed on the outer surface 20 along the length of the tubing 14. The reinforcement structure 22 provides support to the length of tubing 14 to resist kinking of the tubing 14 during surgery.

### CROSS-REFERENCE TO RELATED APPLICATIONS

This application is a continuation-in-part of and claims priority to U.S. patent application Ser. No. 11/186,734, filed Jul. 21, 2005, the entire disclosure of which is incorporated herein by reference.

### Background of the Invention

1. Field of the Invention

The present invention is directed towards surgical tubing. More specifically, the present invention is directed towards surgical tubing for use in ophthalmic surgery for transporting irrigation fluid to a surgical site.

2. Description of Related Art

In surgery, particularly in ophthalmic surgery, the use of irrigation tubing for delivering fluid from an irrigation source to a surgical handpiece and eventually a surgical site is commonly required. In ophthalmic surgery, irrigation fluid is necessary for several reasons, including keeping the eye inflated and preventing collapse of the eye, which can cause serious damage.

It is desirable to have flexible tubing that can expand as fluid is introduced into the tubing, such that the tubing can act as an accumulator during occlusion of such instruments as a phacoemulsification handpiece. In this way, a sufficient supply of irrigating fluid is available quickly to a surgical site upon removal of the occlusion (post occlusion surge) to assist in preventing collapse of the eye. However, one of the significant downsides to very flexible or thin walled tubing is that such tubing is easily kinked which can prevent the flow of fluid into the eye, which is an unacceptable risk.

In order to prevent or at least minimize the change of the tubing kinking and cutting off the supply of fluid, the thickness of the irrigation tubing is often built-up, such that the tubing is rigid or stiff enough to not easily kink. However, the build-up to make the tubing sufficiently stiff to prevent kinking, then may cause an undesirable torque effect on the handpiece that is being used by the surgeon. The weight and the stiffness of the tubing tends to pull on an end of the handpiece, which can fatigue a surgeon's hand as well as continually bias the handpiece in a direction that may not be desired by the surgeon. This may result in the surgeon being uncomfortable during delicate surgery, such as routinely incurred in ophthalmic surgery. Therefore, it would be desirable to have a thin wall irrigation tubing for ophthalmic surgery to act as a fluid accumulator so that sufficient fluid can be supplied to the handpiece to prevent collapse of the eye, but yet still be resistant to bending and kinking, to ensure a constant flow of irrigation fluid into the surgical handpiece.

### BRIEF DESCRIPTION OF THE DRAWING

FIG. 1 is a prior art depiction of thin wall surgical tubing being kinked to prevent the flow of irrigation fluid;

FIG. 2 is a cross-sectional view of irrigation tubing in accordance with the present invention;

FIG. 3 is a partial perspective view of irrigation tubing in accordance with the present invention;

FIG. 4 is a partial elevation view of surgical tubing in accordance with the present invention being bent; and

FIG. 5 is a schematic diagram of a surgical system using tubing in accordance with the present invention.

### DETAILED DESCRIPTION OF THE PREFERRED EMBODIMENT

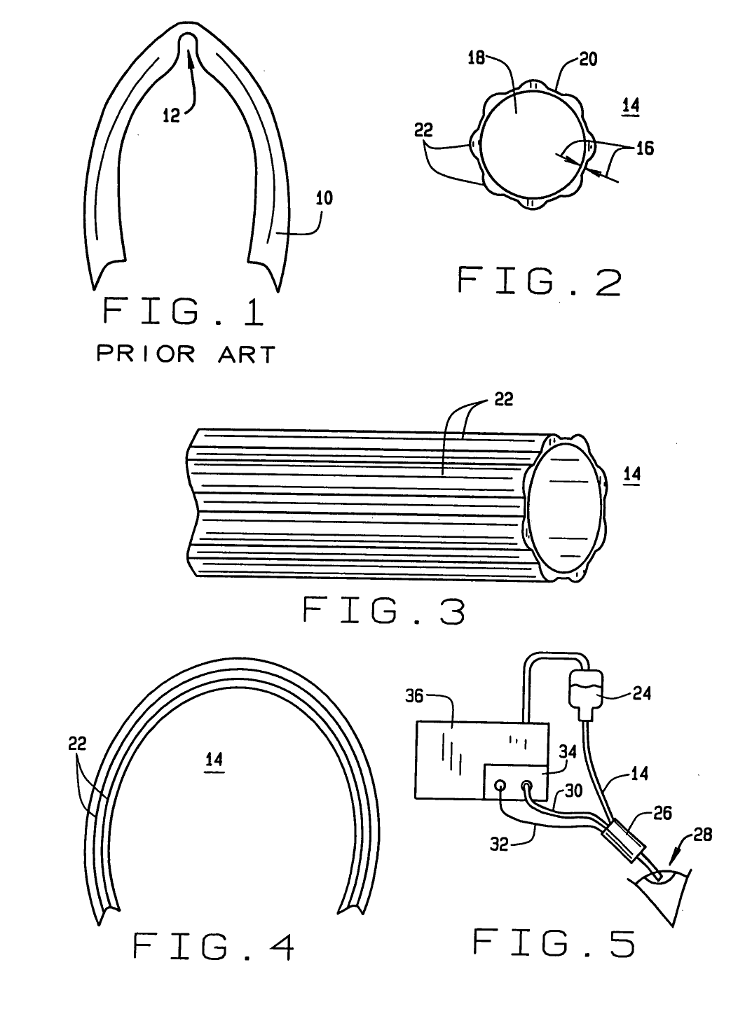

FIG. 1 shows prior art tubing 10 being bent with a kink, shown generally at 12, which causes a flow of fluid through tubing 10 to be cut-off at 12. As discussed above, this is highly undesirable and can lead to significant injury to an eye during surgery. Yet having thin wall tubing is highly desirable in that the tubing can then act as a fluid accumulator during surgery. This ensures that a sufficient supply of fluid is available to a surgical handpiece upon the removal of an occlusion, to help prevent collapse of the eye upon the removal of the occlusion. Historically, there has been a trade-off made between the flexibility of the tubing and the amount of rigidity required to prevent kinking, such that the tubing is flexible enough not to unacceptably torque the handpiece being used by the surgeon but yet be rigid enough not to easily kink.

The present invention, a cross-section of which is shown at FIG. 2, includes a length of surgical irrigation tubing 14 having a desired thickness shown at arrow 16 of approximately 0.015 inches. The inner lumen shown generally at 18 transports fluid from a source to a surgical handpiece. The tubing 14 has an outer surface 20 including reinforcement structure shown at 22 formed on the outer surface 20 along the length of the tubing 14. The reinforcement structure 22 provides support to the length of tubing 14 to resist kinking of the tubing during surgery. Preferably the tubing 14 is formed of silicone, PVC, polyurethane or other acceptable surgical tubing materials.

FIG. 3 shows a partial perspective view of the tubing 14 showing the reinforcement structure as a plurality of raised ribs running along a substantial length of the tubing 14. While the ribs 22 are shown running along the longitudinal axis of the tubing 14, ribs may also run in spirals along tubing 14. It is noted that the reinforcement structure 22 should be formed on the outside of the tubing 14 instead of the inside, so that the reinforcement structure is placed along the larger moment. This provides for greater resistance to bending and therefore, more satisfactorily prevents kinking. Ophthalmic aspiration tubing, as opposed to irrigation tubing, with internal channels or raised ribs is known, such as that shown in published U.S. patent application 2004/0039351 to Barrett. Barrett's tubing is for aspiration and also includes raised ribs on the internal lumen surface, which would provide inferior resistance to bending than the present invention. While the raised ribs 22 have been shown to span the entire length of tubing 14, it is to be understood that the reinforcement structure 22 may not span the entire length of tubing 14, but rather only a substantial length in order to provide sufficient resistance to kinking of the tubing during surgery. Tubing 14 also provides the benefit of being able to receive a standard barb fitting on the end of the tubing for connection to a fluid source or a surgical handpiece.

FIG. 4 shows an elevation view of a partial length of tubing 14 being bent including reinforcement structure 22 that resists bending and therefore prevents or minimizes kinking of the tubing 14 during use in surgery. It is desirable that the thickness of the tubing exclusive of the reinforcement structure 22 be a maximum of 0.015 inches. This is compared to standard prior art irrigation tubing with a thickness of 0.021 inches. By making the thickness of the tubing thin, tubing 14 can act as a fluid accumulator to provide a sufficient supply of fluid quickly to the handpiece upon the removal of an occlusion from the handpiece, such as a phacoemulsification handpiece during surgery. This is quite important, in order to prevent collapse of the eye, which can cause significant and perhaps irreversible damage to the eye (corneal burn). While reinforcement structure 22 has been shown as rounded, raised ribs, those skilled in the art will appreciate that reinforcement structure could take other shapes, such as square-shaped ribs or triangular-shaped ribs or even a series of raised bumps raised in a pattern such that the bumps would provide bending resistance to the tubing 14 or other suitable reinforcement structure.

FIG. 5 shows a length of flexible irrigation tubing 14 having an inner lumen for transporting fluid 24 to a surgical handpiece 26. Surgical handpiece 26 is connected to the tubing 14 for delivering the fluid, shown generally at 28, and wherein the tubing 14 has an outer surface including reinforcement structure, which provides support to the length of tubing, as described above, to resist kinking of the tubing during surgery.

Surgical handpiece 26, is also connected to tubing 30, and power cord 32, which in turn are connected to surgical pump 34 which forms a part of a surgical system 36.

We claim:

1. A length of ophthalmic surgical irrigation tubing comprising:
a length of flexible irrigation tubing having an inner lumen for transporting fluid from a source to a surgical handpiece;
the tubing having an outer surface including reinforcement structure formed on the outer surfaced long the length of tubing wherein the reinforcement structure provides support to the length of tubing to resist kinking of the tubing during surgery.

2. The invention of claim 1, wherein the reinforcement structure is a plurality of raised ribs running along a substantial length of the tubing.

3. The invention of claim 1, wherein the tubing is formed of one of silicone, PVC, or polyurethane.

4. The invention of claim 1, wherein the tubing has a thickness, exclusive of the reinforcement structure of less than 0.015 inches.

5. An ophthalmic surgical system comprising;
a length of flexible irrigation tubing having an inner lumen for transporting fluid from a source to a surgical handpiece;
the surgical handpiece connected to the tubing for performing an ophthalmic surgical procedure, including delivering the fluid to a surgical site; and
wherein the tubing has an outer surface including reinforcement structure formed on the outer surface along the length of tubing and wherein the reinforcement structure provides support to the length of tubing to resist kinking of the tubing during surgery.
